(19) United States
(12) Patent Application Publication
(10) Pub. No.: US 2025/0192467 A1
(43) Pub. Date: Jun. 12, 2025

(54) WIRE HOLDING DEVICE, SYSTEMS, AND METHODS

(71) Applicant: Sterling Innovations, LLC, Scottsdale, AZ (US)

(72) Inventor: Danton Dungy, Scottsdale, AZ (US)

(73) Assignee: Sterling Innovations, LLC, Scottsdale, AZ (US)

(21) Appl. No.: 18/532,820

(22) Filed: Dec. 7, 2023

(51) Int. Cl. H01R 13/502; H01R 13/639; H01R 43/28

(52) U.S. Cl. CPC H01R 13/502; H01R 13/639; H01R 43/28

(57) ABSTRACT

Systems and methods and devices are provided for holding wires, in particular guide wires. In various embodiments, the wire holding device may comprise a cylindrical body configured to hold one or more wires. The wire holding device may comprise a retention tab to hold wires in place and allow for removal of wires. In various embodiments, the retention tab may comprise teeth configured for the wire to be woven between and at least partially held in place. In various embodiments, the wire holding device may be configured to stack. In various embodiments, the wire holding device may comprise protrusions and inserts to prevent rotation when stacked. For example, the wire holding device may comprise male protrusions configured to fit into female inserts to prevent rotation between two or more stacked wire holding devices.

### TECHNICAL FIELD

This disclosure relates to a system, device and method of wire holding, and in particular a wire holding for medical procedures.

### BACKGROUND

In the health, medical, pharmaceutical and/or life science industry, wires may be used for a broad range of applications. In particular, wires, such as guide wires may be used by inserting wires into a vein, vessel or artery to act as a guide to then secure items such as a stent, catheter or other device into place. Additionally, wires may be used in orthodontics to connect members to secure or move teeth placement. Typically, wires, or guide wires are designed to be strategically pushed or steered into place. Therefore, they are both flexible and desire a degree of stiffness which may cause them to be difficult to manage and maintain sterile in surgical environments.

Typically, during medical procedures guide wires are stored by placing wet rags on a table top and sandwiching wires between them. The wet rag works to keep the guide wire in a spiral position and prevent the guide wire from straightening to minimize space and avoid contamination. However, guide wires stored in this manner take up a large amount of space and create risks of contamination. Further, it is difficult to organize. When many guide wires sizes may be required, it may be difficult to ensure the correct wire is used.

Accordingly, there is a need for a system, apparatus and/or method to hold one or more guide wires in a space saving and sterile manner.

### SUMMARY

A system, method and device for holding wires are disclosed. In various embodiments, a method holding and removing one or more wires in a wire holding device is disclosed. The method may comprise providing the wire holding device. In various embodiments, the wire holding device may comprise a cylindrical body consisting of a top, a bottom and a vertical wall, wherein the top and the bottom are at least partially connected by the vertical wall, and a retention tab positioned on a first side of the cylindrical body. The method may comprise inserting a first wire into the cylindrical body of the wire holding device. In various embodiments, the top may comprise one or more panels which define an opening for accessing the cylindrical body. The method may comprise placing the first wire into the retention tab. In various embodiments, the retention tab may be configured to secure the one or more wires in place inside of the cylindrical body.

In various embodiments, a wire holding device is disclosed. In various embodiments, the wire holding device may comprise a bottom and a top each at least partially connected by a vertical wall, which defines a cylindrical body configured to hold one or more wires. In various embodiments, the top may comprise one or more panels which define an opening for accessing the cylindrical body. In various embodiments, the vertical wall may comprise a retention tab configured to hold the one or more wires in place.

In various embodiments, a wire holding system is disclosed. In various embodiments, the system may comprise a cylindrical body consisting of a top, a bottom and a vertical wall. In various embodiments, the top and the bottom may be at least partially connected by the vertical wall, the cylindrical body configured to hold one or more wires. In various embodiments, the system may comprise a retention tab positioned on a first side of the cylindrical body. In various embodiments, the retention tab may be configured to secure the one or more wires in place inside of the cylindrical body.

### BRIEF DESCRIPTION OF THE DRAWINGS

Other systems, methods, features, and advantages of the present invention will be apparent to one skilled in the art upon examination of the following figures and detailed description. Component parts shown in the drawings are not necessarily to scale, and may be exaggerated to better illustrate the important features of the present invention.

FIG. 1 illustrates a side perspective view of a wire holding device, in accordance with various embodiments;

FIG. 2 illustrates a perspective view of a retention tab of a wire holding device, in accordance with various embodiments;

FIG. 3 illustrates a side perspective view of a stacking system for one or more wire holding devices, in accordance with various embodiments; and

FIG. 4 illustrates a flowchart for a method of holding wires in a device, in accordance with various embodiments.

### DETAILED DESCRIPTION

For simplicity and clarity of illustration, the drawing figures illustrate the general manner of construction, and descriptions and details of some features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

Disclosed herein are systems, devices and/or methods for holding wires, such as guide wires. In various embodiments, a wire holding device may be used to hold or secure one or more wires. For example, the wire holding device may resemble a frisbee or disk like shape. The cylindrical wire holding device may comprise a cylindrical body which may hold one or more wires. The wire holding device may have a top with an opening to allow access to the cylindrical body for wires to be placed inside. The wire holding device, in various embodiments, may comprise a retention tab, which holds the wire in place and allows a user to pull a portion or the entire wire from the wire holding device. The retention tab may comprise teeth, which allow a wire to be woven through and held in place.

In various embodiments, the wire holding device may be configured to stack. For example, wire holding devices may be configured to be stacked one on top of another. Further, the wire holding device may have anti rotation components, such as protrusions and inserts that fit together, to allow the wire holding devices to neatly stack and prevent rotation. In various embodiments, the wire holding device may comprise protrusions or inserts on either the top or bottom to prevent rotation when wire holding devices are stacked. In various embodiments, the anti rotation components may be positioned so that when the wire holding devices are stacked, the retention tabs are aligned. In various embodiments, the anti rotation components, such as the anti rotation protrusion and anti rotation inserts may be rectangular shaped. In various embodiments, the anti rotation components, such as the anti rotation protrusion and anti rotation inserts may be dimples or divots on the top and bottom of the wire holding device. The anti rotation protrusion and anti rotation inserts may be a suitable structure to prevent rotation and reduce movement when wire holding devices are stacked.

In various embodiments, the wire holding device may be disposable and designed for single use. In various embodiments, the wire holding device may be single use to ensure it is sterile when wires are inserted. In various embodiments, the guide wire device may comprise a label on the side which can be seen when placed on a table or when multiple wire holding devices are stacked. The label may be used to display the wire size or date or other information to identify the contents of the wire holding device. In various embodiments, the wire holding device may each be configured to hold two or more wires. In various embodiments, the wire holding device may hold multiple wires by weaving them in different locations of the teeth.

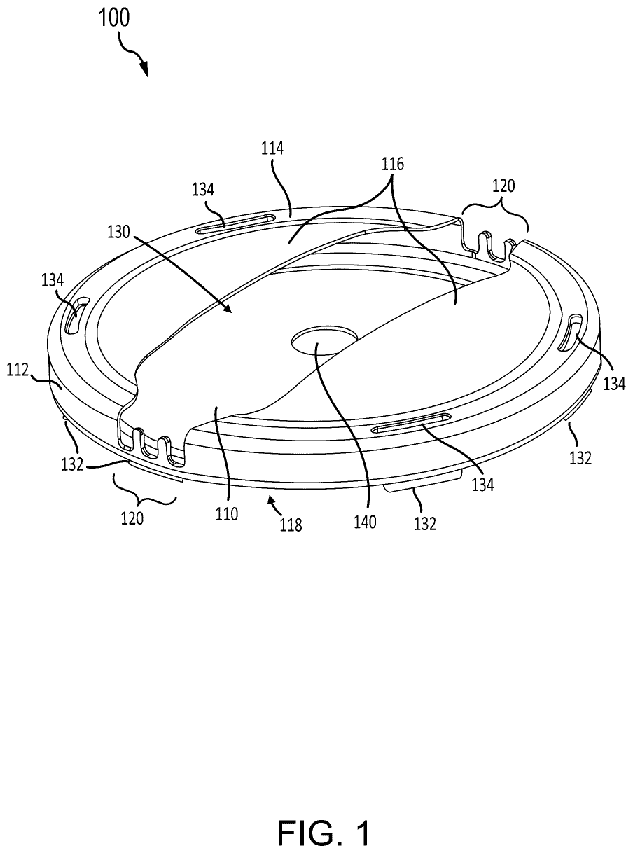

With respect to FIG. 1, a wire holding device 100 is shown, in accordance with various embodiments. In various embodiments, the wire holding device 100 may be configured to hold wires and optionally secure wires in place. In various embodiments, the wire holding device 100 may be made primarily of plastic, synthetic or other materials. In various embodiments, the wire holding device 100 may be made of one solid piece or multiple pieces bonded or attached together. In various embodiments, the wire holding device 100 may hold guide wires of various lengths and sizes. In various embodiments, the wire holding device 100 may resemble a frisbee and may easily be moved or transported. In various embodiments, the wire holding device 100 may be placed on a table to allow for easy assess during surgery or other procedures.

In various embodiments, the wire holding device 100 may comprise a cylindrical body 110. In various embodiments, the cylindrical body 110 may be at lease partially cylindrical, or any suitable shape to hold wires. In various embodiments, the cylindrical body 110 may be a hollow interior of the wire holding device 100. In various embodiments, the cylindrical body may be defined by a top 114 a bottom 118, and vertical wall 112. For example, the bottom 118 and the top 114 may be connected at least partially by a vertical wall 112 to define a cylindrical body 110.

In various embodiments, the top 114 and the bottom 118 may be circular shaped. For example, the top 114 and bottom 118 may be circular, wherein when they are connected with the vertical wall 112, they are substantially cylindrical. In various embodiments, the cylindrical body 110 may be cylindrically shaped to allow for wires to be coiled/spiraled and easily inserted and held in place a compact position. In various embodiments, the bottom 118 may be placed on a table and the vertical wall 112 extends upwards and connects with the top 114.

In various embodiments, the top 114 may comprise an opening to allow access to the cylindrical body 110. For example, the top 114 may comprise flaps 116. The flaps 116 may extend radially inward towards the center of the wire holding device 100. In various embodiments, the top 114 may partially cover the top of the cylindrical body 110. In various embodiments, the flaps 116 may define an opening 130. In various embodiments, the flaps 116 may extend inward to allow for a hand or device to be inserted into the opening 130 of the wire holding device 100. For example, the flaps 116 may partially cover the top of the cylindrical body 110 to allow a wire to be inserted through the opening 130 and into the cylindrical body 110. In various embodiments, the flaps 116 may be connected to the vertical wall 112 and comprise an opening 130 of at least 6 cm and less than 20 cm. In various embodiments, the wire holding device 100 may be between 20 cm and 25 cm in width and approximately 25 mm in height.

In various embodiments, the wire holding device 100 may comprise one or more retention tabs 120. For example, the wire holding device 100 may comprise a retention tab 120 positioned on the side or vertical wall 112. In various embodiment, the retention tab 120 may be positions in line with the opening 130. For example, in various embodiments, the retention tab 120 may be positioned adjacent to each of the flaps 116. In various embodiments, the wire holding device may comprise two retention tabs 120. In various embodiments, the retention tabs 120 may be positioned on opposite ends of the wire holding device 100.

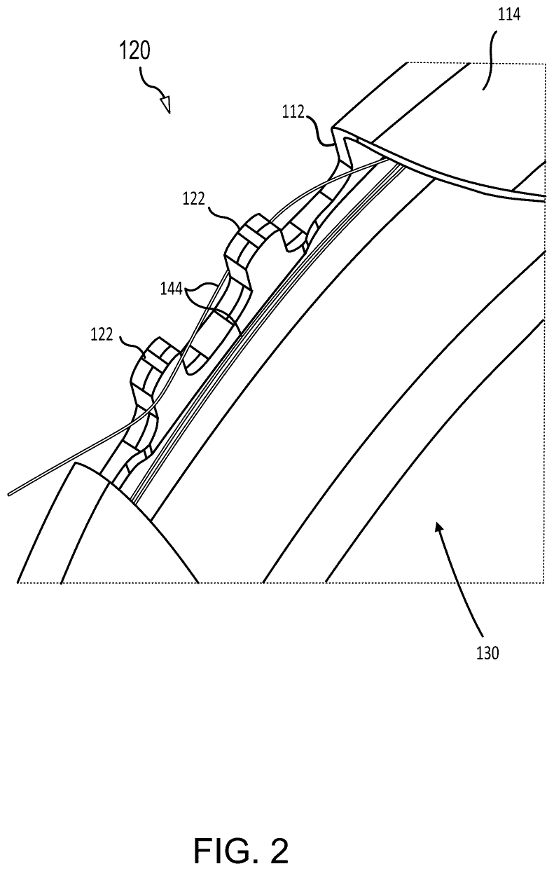

In various embodiment, the retention tab 120 may be configured to hold or secure one or more wires in place, as described with reference to FIG. 2. With reference back to FIG. 1, the retention tab 120 may be positioned in line with the opening 130, such that when a wire is inserted into the opening 130 may be easily placed into the retention tab 120.

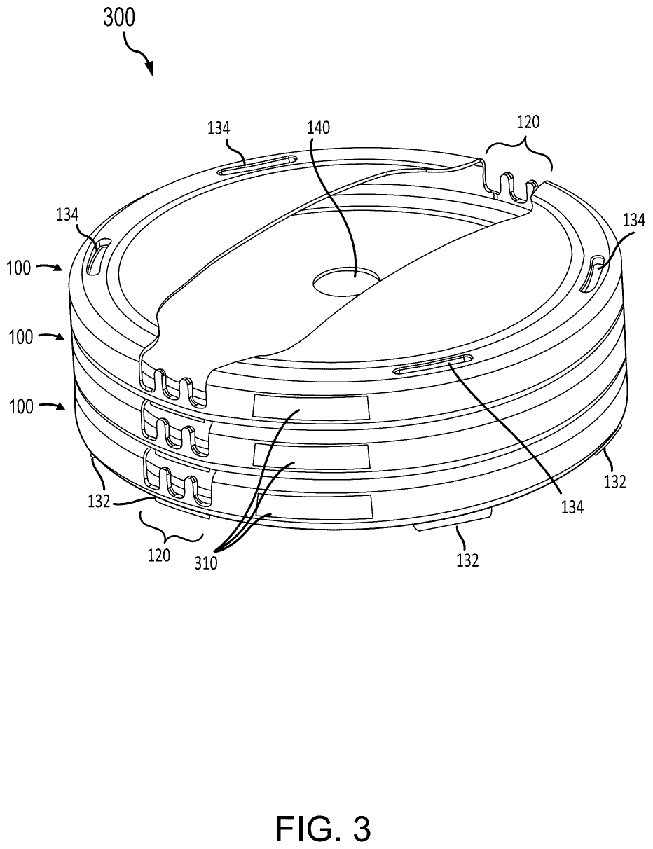

In various embodiments, the wire holding device 100 may comprise one or more anti rotation protrusions 132 and/or anti rotation inserts 134, as described in greater detail with reference to FIG. 3. With reference back to FIG. 1, the anti rotation protrusions 132 may be positioned on the bottom 118 of the wire holding device 100. In various embodiments, the anti rotation protrusions 132 may extend at least partially outward from the bottom 118. In various embodiments, the anti rotation protrusions may be positioned on the bottom 118 to raise or level the wire holding device 100 when placed on a surface. In various embodiments, the anti rotation protrusions 132 may be evenly spaced on the bottom 118. In various embodiments, anti-rotation protrusions 132 may be spaced to provide adequate support for the wire holding device 100. In various embodiments, the wire holding device 100 may comprise three or more anti rotation protrusions 132. In various embodiments, the wire holding device 100 may comprise four anti rotation protrusions 132. In various embodiments, the anti rotation inserts 134 may be positioned on the top 114 of the wire holding device 100. In various embodiments, the anti rotation inserts 134 may comprise a channel or ridge. In various embodiments, the anti rotation inserts 134 may extend at least partially inward into the wire holding device 100. In various embodiments, the anti rotation inserts 134 may be configured to receive a anti rotation protrusion 132. In various embodiments, the anti rotation protrusions 132 may be at least 2 mm thick to prevent breakage. In various embodiments, the depth of the anti rotation inserts 134 may be equal to or greater than the height of the anti rotation protrusions 132. In various embodiments, the wire holding device 100 may comprise at least four anti rotation protrusions 132 and four anti rotation inserts 134, to allow the wire holding devices 100 to be secure when stacked. In various embodiments, the anti rotation inserts 134 may be wells, dimples, a channel or channels, or any suitable means to secure with the anti rotation protrusions 132.

In various embodiments, the anti rotation protrusions 132 may vary in height to balance the wire holding device 100. For example, three of the anti rotation protrusions 132 may be a first height and three of the anti rotation protrusions 132 may be a second height, wherein the first height is less than the second height. For example, three of the anti rotation protrusions 132 may extend to a first height of approximately 3 mm and three of the anti rotation protrusions 132 may extend to a second height of approximately 2 mm. In various embodiments, the anti rotation protrusions 132 may both be configured to fit with anti rotation inserts 134 or be used to balance the wire holding device 100 on a table. In various embodiments, the anti rotation inserts 134 and anti rotation protrusions 132 may secure to prevent malalignment and side to side movement.

In various embodiments, the wire holding device 100 may further comprise a center hole 140. For example, the center hole 140 may be positioned at the center axis of the wire holding device 100. In various embodiments, the center hole 140 may be approximately 3 cm. In various embodiments, the center hole 140 may allow a finger to easily be inserted to pick up the wire holding device 100. In various embodiments, the center hole 140 may be aligned with the opening 130. The center hole 140 may be configured to allow a rod or pole to be inserted in the center of one or more wire holding devices 100.

In various embodiment, the wire holding device 100 may comprise a liquid. For example, the cylindrical body 110 may be configured to hold a liquid. In various embodiments, the wire holding device may be configured to hold approximately 20 cc of liquid. In various embodiments, the liquid may be configured to lubricate the wires. In various embodiments, the liquid may clean the wires with saline, water, antibiotics, gel, etc.

With reference now to FIG. 2, a zoomed in view of a retention tab 120 of a wire holding device 100 is shown, in accordance with various embodiments. In various embodiments, the retention tab 120 may comprise one or more teeth 122. The teeth may extent from the bottom 118 of the wire holding device 100. For example, the teeth 122 may extend upward and perpendicular to the bottom 118. In various embodiments, the teeth 122 may be positioned between portions of the vertical wall 112. For example, the vertical wall 112 may comprise one or more portions which connect the top 116 to the bottom 118, wherein the retention tab 120 is positioned between the connecting portions of the vertical wall 112. In various embodiments, the teeth 122 may be thicker than the thickness of the vertical wall 112, as shown in FIG. 2. For example, the teeth 122 may be at least 1 mm thicker than the vertical wall 112 to allow the wires to more easily be secured in place. In various embodiments, the wire holding device 100 may be configured to hold a wire 144. For example, the wire 144 may be woven between the retention tab 120. In various embodiments, the retention tab 120 may comprise two teeth 122. For example, the wire 144 may be woven through the teeth 122 to secure the wire in place. In various embodiments, the teeth 122 may be spaced at least 3 mm from each other to allow a wire 144 to be placed between them. In various embodiments, a wire 144 may be place in the retention tab 120 such that the wire is threaded out of the first teeth 122 and into the second teeth 122 and outside of the vertical wall 112. The retention tab 120 may be connected on one end to the bottom 118 and the teeth 122 may extend radially upward.

With reference now to FIG. 3, a stacking system 300 for one or more wire holding devices 100 is shown, in accordance with various embodiments. As described with reference to FIG. 1 and shown in greater detail in FIG. 3, the wire holding device 100 may comprise one or more anti rotation protrusion 132 and one or more anti rotation inserts 134. The stacking system 300 may comprise two or more wire holding devices 100 stacked on top of each other. In various embodiments, the anti rotation inserts 134 may be configured to receive the anti rotation protrusion 132. The anti rotation protrusion 132 and anti rotation inserts may prevent rotation between the wire holding devices 100 when stacked in the stacking system 300.

In various embodiments, the wire holding device 100 may comprise six anti rotation protrusions 132 and four anti rotation inserts 134. In various embodiments, when the wire holding devices 100 are stacked, four of the anti rotation protrusions 132 may fit into the four anti rotation inserts 134, and two of the anti rotation protrusions 132 may be positioned between the opening 130 and above the retention tab 120.

In various embodiments, the wire holding device 100 may further comprise a label 310. The label 310 may be positioned on the outside of the wire holding device 100. For example, the label 310 may be positioned on the vertical wall 112. In various embodiments, when the wire holding device 100 is stacked using the anti rotation protrusions 132 and anti rotation inserts 134, the labels 310 may align to allow a user to easily read the label. The label 310 may be used to show important information, such as the date and/or wire size.

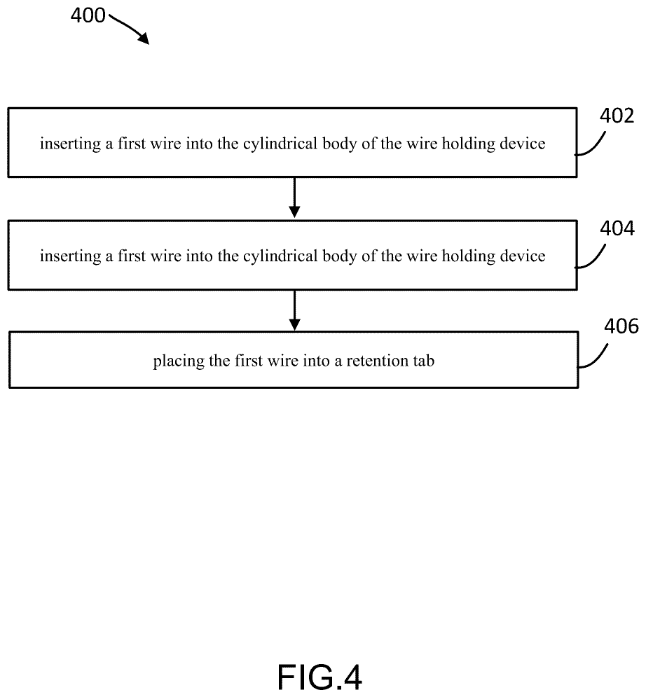

With reference now to FIG. 4, a method of holding wires in a device 400 is shown, in accordance with various embodiments. The method 400 may comprise providing the wire holding device (402). In various embodiments, the wire holding device may comprise a cylindrical body consisting of a top, a bottom and a vertical wall. In various embodiments, the top and the bottom are at least partially connected by the vertical wall, and a retention tab positioned on a first side of the cylindrical body. The method 400 may further comprise inserting a first wire into the cylindrical body of the wire holding device (404). In various embodiments, the top comprises one or more panels which define an opening for accessing the cylindrical body. The method 400 may further comprise placing the first wire into the retention tab (406). In various embodiments, the retention tab may be configured to secure the one or more wires in place inside of the cylindrical body.

Exemplary embodiments of the methods/systems have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A wire holding device comprising:
a bottom and a top each at least partially connected by a vertical wall, which defines a cylindrical body configured to hold one or more wires;
wherein the top comprises one or more panels which define an opening for accessing the cylindrical body; and
wherein the vertical wall comprises a retention tab configured to hold the one or more wires in place.

2. The device of claim 1, wherein the wire holding device comprises one or more anti-rotation protrusions.

3. The device of claim 2, wherein the wire holding device comprises one or more anti rotation inserts configured to receive the anti-rotation protrusions and prevent rotation when two or more wire holding devices are stacked.

4. The device of claim 1, wherein cylindrical body comprises a fluid configured to lubricate the wires.

5. The device of claim 1, wherein the retention tab comprises one or more teeth to hold the one or more wires in place.

6. The device of claim 5, wherein the teeth of the retention tab are thicker than the vertical wall, and wherein the teeth are configured for the one or more wires to be woven between each of the teeth.

7. The device of claim 1, wherein the vertical wall comprises a second wire retention tab positioned on the opposite side of the cylindrical body and comprising one or more teeth to hold the one or more wires in place.

8. A wire holding system comprising:
a cylindrical body consisting of a top, a bottom and a vertical wall, wherein the top and the bottom are at least partially connected by the vertical wall, the cylindrical body configured to hold one or more wires; and
a retention tab positioned on a first side of the cylindrical body;
wherein the retention tab is configured to secure the one or more wires in place inside of the cylindrical body.

9. The system of claim 8, wherein the top of the cylindrical body comprises one or more panels which define an opening for accessing the cylindrical body.

10. The system of claim 8, further comprising one or more anti-rotation protrusions attached to the bottom of the cylindrical body.

11. The system of claim 10, further comprising one or more anti rotation-inserts positioned on the top of the cylindrical body and configured to receive the anti-rotation protrusions and prevent rotation when the wire holding system is stacked.

12. The system of claim 8, wherein cylindrical body comprises a fluid configured to lubricate the wires.

13. The system of claim 8, wherein the retention tab comprises one or more teeth to hold the one or more wires in place.

14. The system of claim 13, wherein the teeth of the retention tab are thicker than the vertical wall, and wherein the teeth are configured for the one or more wires to be woven between each of the teeth.

15. The system of claim 8, wherein the vertical wall comprises a second wire retention tab positioned on the opposite side of the cylindrical body and comprising one or more teeth to hold the one or more wires in place.

16. A method of holding and removing one or more wires in a wire holding device comprising:
providing the wire holding device;
the wire holding device comprising a cylindrical body consisting of a top, a bottom and a vertical wall, wherein the top and the bottom are at least partially connected by the vertical wall, and a retention tab positioned on a first side of the cylindrical body;
inserting a first wire into the cylindrical body of the wire holding device;
wherein the top comprises one or more panels which define an opening for accessing the cylindrical body; and
placing the first wire into the retention tab;
wherein the retention tab is configured to secure the one or more wires in place inside of the cylindrical body.

17. The method of claim 16 further comprising:
removing the first wire from the wire holding device by pulling the first wire away from the retention tab.

18. The method of claim 16 further comprising:
stacking the wire holding device on a second wire holding device, wherein the wire holding device comprises one or more anti-rotation protrusions and the second wire holding device comprises one or more anti rotation-inserts configured to receive the anti-rotation protrusions and prevent rotation when the wire holding devices and the second wire holding device is stacked.

19. The method of claim 16, further comprising:
weaving the first wire between one or more teeth of the retention tab to partially secure the first wire in place.

20. The method of claim 19, wherein the one or more teeth of the retention tab are thicker than the vertical wall.
